(19) United States
(12) Patent Application Publication
(10) Pub. No.: US 2023/0260603 A1
(43) Pub. Date: Aug. 17, 2023

(54) MACHINE LEARNING SYSTEMS AND METHODS FOR POURBAIX DIAGRAM DESCRIPTOR-BASED PREDICTION OF ELETROCHEMICAL FIGURES OF MERIT

(71) Applicant: Toyota Research Institute, Inc., Los Altos, CA (US)

(72) Inventors: Joseph Harold Montoya, Berkeley, CA (US); Santosh K. Suram, Mountain View, CA (US)

(21) Appl. No.: 17/670,736

(22) Filed: Feb. 14, 2022

(51) Int. Cl. G16C 20/70; G06N 20/00; G16C 20/30

(52) U.S. Cl. CPC G16C 20/70; G06N 20/00; G16C 20/30

(57) ABSTRACT

A system for predicting an electrochemical figure of merit for a material in contact with a liquid includes a processor and a memory communicably coupled to the processor. The memory stores machine-readable instructions that, when executed by the processor, cause the processor to embed an input dataset in a feature space of a machine learning module. The input dataset includes a material representation with a material composition, electrochemical parameters from a Pourbaix diagram, and chemical species of the material composition in contact with the liquid. The memory also stores machine-readable instructions that, when executed by the processor, cause the processor to predict, based at least in part on the input dataset embedded in the feature space, an electrochemical figure of merit for the material representation exposed to the liquid.

### TECHNICAL FIELD

The present disclosure relates generally to machine learning of material properties and particularly to machine learning of electrochemical figures of merit.

### BACKGROUND

Predicting kinetic properties, such as corrosion rate and/or catalytic activity, for materials exposed to a liquid (e.g., aqueous) environment, is an ongoing challenge for scientists and engineers. Particularly, known kinetic property values or data are typically experimentally determined and prediction of such properties for different materials and/or different environmental conditions is often unsuccessful. In contrast, thermodynamic data, i.e., equilibrium data, for materials and for materials exposed to different environmental conditions are more common. For example, Pourbaix diagrams are phase diagrams that map conditions of equilibrium potential and acidity/basicity (i.e., pH) for stable chemical species of a material exposed to a liquid environment. Stated differently, a Pourbaix diagram is a plot of electrochemical stability as a function of pH for different oxidation-reduction (redox) states of one or more elements exposed to a liquid at a particular temperature and pressure. In addition, Pourbaix diagrams provide a large volume of thermodynamic information in an efficient compact format. However, Pourbaix diagrams do not provide information on kinetic properties of the one or more elements exposed to the liquid.

The present disclosure addresses issues related to using Pourbaix diagrams to predict kinetic properties, and other issues related to Pourbaix diagrams.

### SUMMARY

This section provides a general summary of the disclosure and is not a comprehensive disclosure of its full scope or all of its features.

In one form of the present disclosure, a system for predicting an electrochemical figure of merit (EFOM) includes a processor and a memory communicably coupled to the processor. The memory stores machine-readable instructions that, when executed by the processor, cause the processor to embed an input dataset in a feature space of a machine learning module. The input dataset includes a material representation comprising a material composition, electrochemical parameters from a Pourbaix diagram for the material composition in contact with a liquid, and chemical species of the material composition in contact with the liquid. The memory also stores machine-readable instructions that, when executed by the processor, cause the processor to predict, based at least in part on the input dataset embedded in the feature space, an EFOM for the material representation.

In another form of the present disclosure, a system includes a processor and a memory communicably coupled to the processor. The memory stores an acquisition module and a machine learning module that includes instructions that, when executed by the processor cause, the processor to select an input dataset from a candidate dataset and Pourbaix diagram from a Pourbaix diagram dataset. The input dataset includes material representations comprising a material composition, electrochemical parameters from the Pourbaix diagram, and chemical species of the material composition in contact with an aqueous solution represented in the Pourbaix diagram. The acquisition module and the machine learning module also include instructions that when executed by the processor cause the processor to: (1) embed the input dataset in a feature space of the machine learning module; (2) train a machine learning model during one or more iterations to predict, based at least in part on the input dataset embedded in the feature space, an electrochemical figure of merit for the material representations in the input dataset; and (3) predict, based at least in part on the trained machine learning model, the EFOM for a material representation not in the input dataset.

In still another form of the present disclosure, a method includes embedding an input dataset in a feature space of a machine learning module with the input dataset including a material representation comprising a material composition, electrochemical parameters from a Pourbaix diagram for the material composition in contact with a liquid, and chemical species of the material composition in contact with the liquid. The method also includes predicting, based at least in part on the input dataset embedded in the feature space, an EFOM for another material representation not in the input dataset.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description and the accompanying drawings, wherein:

FIG. 1 shows a Pourbaix diagram for the iron-oxygen-hydrogen system at a temperature of 298 K and a pressure of 1 atm;

FIG. 2 shows a Pourbaix diagram for the vanadium-oxygen-hydrogen system at a temperature of 298 K and a pressure of 1 atm;

FIG. 3 shows a Pourbaix diagram for the iron-vanadium-oxygen-hydrogen system at a temperature of 298 K and a pressure of 1 atm;

FIG. 4 illustrates an example of a machine learning system that uses a Pourbaix diagram to predict an electrochemical figure of merit for a material composition according to the teachings of the present disclosure;

FIG. 5 shows a flow chart for a method using the machine learning system illustrated in FIG. 4 to predict an electrochemical figure of merit according to the teachings of the present disclosure;

FIG. 6 shows a flow chart for another method using the system illustrated in FIG. 4 to predict an electrochemical figure of merit according to the teachings of the present disclosure; and

FIG. 7 shows a diagram illustrating the methods in FIGS. 5 and 6.

### DETAILED DESCRIPTION

The present disclosure provides a machine learning (ML) system and a ML method for predicting an EFOM for a material composition (also referred to herein simply as “material”) exposed to a liquid. Particularly, material representations of the material, including Pourbaix diagram information such as thermodynamic data and/or chemical species of the material exposed to the liquid, are embedded in a feature space of a ML module and used for training a ML model. And after training of the ML model, prediction of an EFOM for the material exposed to the liquid is provided.

As used herein, phrase “electrochemical figure of merit” and the term “EFOM” refer to a kinetic property of the material such as but not limited to corrosion rate, catalytic activity, and discharge rate. Also, as used herein, the phrase “corrosion rate” refers to the rate (speed) at which a material in a particular environment deteriorates (corrodes), the phrase “catalytic activity” refers to a rate of a predefined chemical reaction caused by a catalyst material, and the phrase “discharge rate” refers to a steady electrical current from or through a material in electrical connection with a battery of predefined capacity and over a predefined time period.

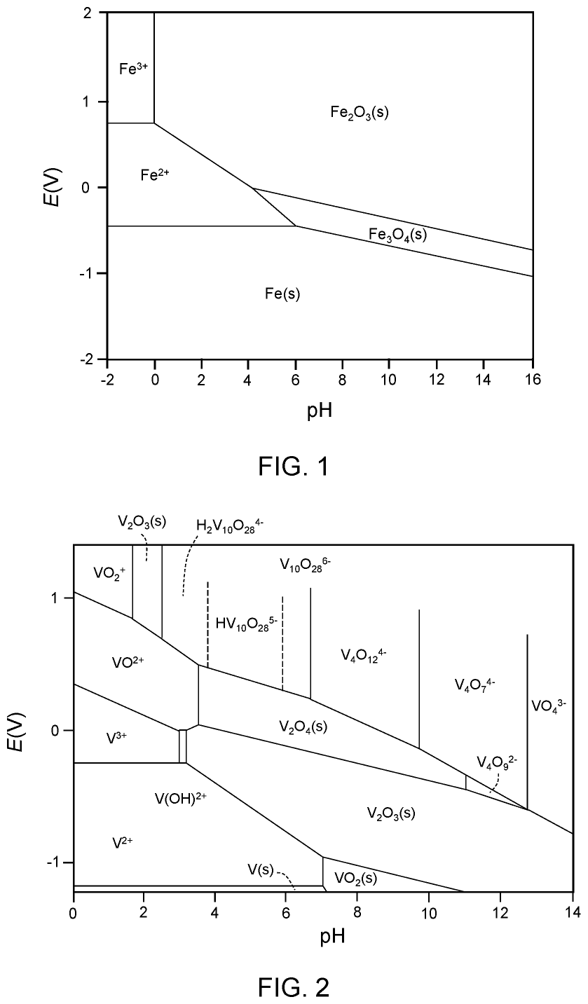

Referring to FIG. 1, a Pourbaix diagram for pure iron (Fe) in contact with an infinite reservoir of an aqueous solution is shown. Also, the Pourbaix diagram shows the possible thermodynamically stable phases or chemical species of Fe in contact with the aqueous solution as a function of electric potential ‘E’ applied to the Fe (also referred to herein simply as “applied potential” or “potential”) and pH of the aqueous solution. As used herein, the term “pH’ is equal to −log₁₀ c, where c is the hydrogen concentration in moles per liter. Accordingly, the phase region labeled ‘Fe” corresponds to conditions of applied potential and pH (of the aqueous solution) where elemental Fe is thermodynamically stable (i.e., does not corrode) in the aqueous solution. In contrast, the phase region labeled ‘Fe²⁺’ corresponds to conditions of applied potential and aqueous solution pH where elemental Fe dissolves (i.e., corrodes) as Fe²⁺ ions in the aqueous solution and the phase region labeled ‘Fe³⁺’ corresponds to conditions of applied potential and aqueous solution pH where elemental Fe dissolves as Fe³⁺ ions in the aqueous solution. And regarding the phase regions labeled Fe₃O₄(s) and Fe₂O₃(s), these phase regions correspond to conditions of applied potential and aqueous solution pH where elemental Fe reacts with oxygen in the aqueous solution to form the Fe-oxides Fe₃O₄ and Fe₂O₃, respectively. Stated differently, Fe₃O₄ and Fe₂O₃ are thermodynamically stable phases when elemental Fe has an electric potential applied thereto and is in contact with an aqueous solution with pH values corresponding to or falling within the phase regions labeled Fe₃O₄ and Fe₂O₃, respectively. And while the Fe—O—H system shown in the Pourbaix diagram in FIG. 1 has only five (5) phase regions, other systems are more complex as discussed below.

Referring to FIG. 2, a Pourbaix diagram for vanadium (V) in contact with an infinite reservoir of an aqueous solution is shown in FIG. 2. And as observed from the figure, vanadium has fifteen (15) possible thermodynamically stable phases depending on the potential applied to elemental V and the pH of the aqueous solution. In addition, and as explained with respect to FIG. 1 above, each phase region in FIG. 2 corresponds to conditions (i.e., applied potential and aqueous solution pH) for which a given (labeled) phase is thermodynamically stable when in contact with the aqueous solution.

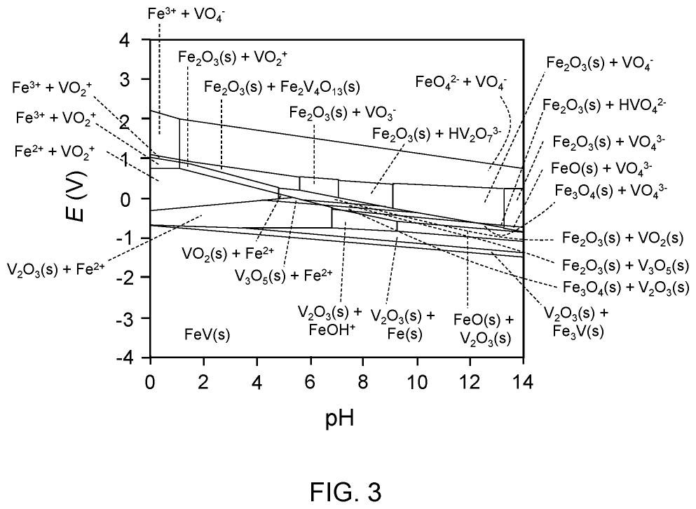

Referring to FIG. 3, a Pourbaix diagram for an even more complex system is shown. Particularly, the Pourbaix diagram for Fe and V in contact with an infinite reservoir of an aqueous solution is shown in FIG. 3. The Pourbaix diagram assumes, i.e., calculation of the Pourbaix diagram assumes, a 1:1 composition (atom fraction) of Fe and V, ionic concentrations of 10⁻⁵ molar concentration (M) of Fe and V ionic species when present, an activity of 1 for solids, a temperature of 298 K and a pressure of 1 atm (e.g., see Electrochemical Stability of Metastable Materials, Singh et al., Chem. Mater. 2017, 29, 10159-10167). And as observed from the figure, the Fe—V—O—H systems has twenty-seven (27) thermodynamically stable phase regions, and fifteen (15) thermodynamically solid phases, depending on the potential applied to elemental vanadium and the pH of the aqueous solution.

It should be understood that thermodynamic data for the redox reactions between chemical species on the Pourbaix diagram can be obtained from the Nernst equation. For example, and using the Fe—V system for illustrative purposes, the Fe—V system results in redox reactions such as:

aFe(s)+bV(s)+cH₂O(l)=>FeₐV_{b}O_{c}H_{d}^{m}(aq)+(2c−d)H⁺(2c−d+m)e⁻

where a, b, c, and d are the stoichiometric coefficients of Fe, V, O, and H, respectively, and m is the charge on the aqueous species FeₐV_{b}O_{c}H_{d}^{m} which is positive, negative, or zero. And at equilibrium, the Nernst equation can be used to relate the applied potential (E) to the reaction Gibbs free energy, ΔᵣG, for each possible redox reaction. Particularly, the reaction Gibbs energy is related to the equilibrium potential E^{o} and the activity quotient ‘Q’ of a redox reaction according to the equation:

−vFE^{o}=ΔᵣG=ΔᵣG^{o}+RT ln Q Eq. 1

where T is the temperature, F is the Faraday constant, R is the ideal gas constant, and the activity quotient Q of the redox reaction above is:

$$Q = \frac{{( {a}_{P} )}^{p} \cdot {( {a}_{H +} )}^{h}}{{( {a}_{R} )}^{r} \cdot {( {a}_{H 2 O} )}^{w}} \qquad Eq . 2$$

such that Eqn. 1 becomes:

$$- υ {FE}^{o} = {Δ}_{r} G = {Δ}_{r} {G}^{o} + RT ln [ \frac{{( {a}_{P} )}^{p} \cdot {( {a}_{H +} )}^{h}}{{( {a}_{R} )}^{r} \cdot {( {a}_{H 20} )}^{w}} ] \qquad Eq . 3$$

$$= {Δ}_{r} {G}^{o} + 2.303 RT ln [ \frac{{( {a}_{P} )}^{p}}{{( {a}_{R} )}^{r} \cdot {( {a}_{H 2 O} )}^{w}} ] - 2.3 \; 03 h RTp H \qquad Eq . 4$$

where ν=(2c−d+m) is the number of electrons, a_{R}^{r}=a_{Fe(s)}^{a}a_{V(s)}^{b} is the activity of the reactants, a_{P}^{p}=a_{Fe}_{ₐ}_{V}_{_{b}}_{O}_{_{c}}_{H}_{_{d}}_{^{m}} is the activity of the products, a_{H2O}^{w}=a_{H2O}^{c} is the activity of water, and a_{H}_{⁺}^{h}=a_{H}_{⁺}^{(2c-d)} is the activity of hydrogen ions for the redox reaction above.

Accordingly, the Pourbaix diagram provides thermodynamically stable phases, including stable chemical species, which can be present when a material is contact with a liquid, and the Nernst equation provides thermodynamic data (e.g., ΔᵣG) for the material (and chemical species) in contact with the liquid as a function of potential applied to the material and the pH of the liquid. In addition, metastable chemical species that can be present when the material is contact with the liquid can be determined and plotted on a Pourbaix diagram as taught in the publication Electrochemical Stability of Metastable Materials, Singh et al., Chem. Mater. 2017, 29, 1 0159-10167, which is disclosed herein in its entity by reference. That is, metastable chemical species that can be present when the material is contact with the liquid can be derived from the Pourbaix diagram.

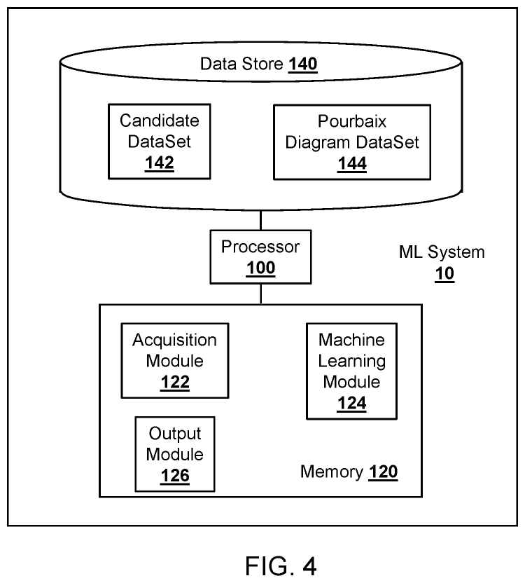

Referring now to FIG. 4, a ML system 10 for predicting an EFOM for one or more materials is illustrated. The ML system 10 is shown including at least one processor (referred to herein simply as “processor”) 100, and a memory 120 and a data store 140 communicably coupled to the processor 100. It should be understood that the processor 100 can be part of the ML system 10, or in the alternative, the ML system 10 can access the processor 100 through a data bus or another communication path.

The memory 120 is configured to store an acquisition module 122, a ML module 124, and an output module 126. The memory is a random-access memory (RAM), read-only memory (ROM), a hard-disk drive, a flash memory, or other suitable memory for storing the acquisition module 122, ML module 124, and output module 126. Also, the acquisition module 122, ML module 124, and output module 126 are, for example, computer-readable instructions that when executed by the processor 100 cause the processor to perform the various functions disclosed herein.

In some variations the data store 140 is a database, e.g., an electronic data structure stored in the memory 120 or another data store. Also, in at least one variation the data store 140 in the form of a database is configured with routines that can be executed by the processor 100 for analyzing stored data, providing stored data, organizing stored data, and the like. Accordingly, in some variations the data store 140 stores data used by the acquisition module 122, the ML module 124, and/or the output module 126. For example, and as shown in FIG. 4, in at least one variation the data store stores a candidate dataset 142 and a Pourbaix diagram dataset 144. In some variations the candidate dataset 142 includes a listing of a plurality of materials and the Pourbaix diagram dataset 144 includes a Pourbaix diagram for one or more of the plurality of materials listed in the candidate dataset 142. And in at least one variation, the candidate dataset 142 includes a training dataset with one or more materials tagged with one or more EFOMs.

Non-limiting examples of materials in the candidate dataset 142 include polymers, metals, intermetallics, semiconductors, insulators, ceramics, and combinations thereof (e.g., ceramic matrix composites and metal matrix composites, among others). The Pourbaix diagram dataset 144 includes a Pourbaix diagram with thermodynamically stable chemical species for a corresponding material in the candidate dataset 142 and/or a tabulated representation of the Pourbaix diagram. And in at least one variation, the Pourbaix diagram includes metastable chemical species for a corresponding material in the candidate dataset 142 and/or a tabulated representation of the Pourbaix diagram.

The acquisition module 122 can include instructions that function to control the processor 100 to select a material from the candidate dataset 142, a corresponding Pourbaix diagram from the Pourbaix diagram dataset 144, and one or more material representations from the Pourbaix diagram. For example, in some variations the acquisition module 122 can include instructions that function to control the processor 100 to select a potential, or a range of potentials, a pH, or a range of pH, and one or more stable and/or metastable chemical species from a given or predefined phase field of the selected Pourbaix diagram. And in at least one variation the acquisition module 122 can include instructions that function to control the processor 100 to provide the selected potential or range of potentials, the selected pH or range of pH, and the selected one or more stable and/or metastable chemical species as an input dataset to the ML module 124. In such variations, the ML module 124 can include instructions that function to control the processor 100 to embed the input dataset in a feature space, e.g., the input dataset can be configured as a feature vector and the feature vector is embedded in the feature space.

The ML module 124 includes instructions that function to control the processor 100 to train a ML model (algorithm) using the embedded input dataset. In some variations, the ML module 124 includes instructions that function to control the processor 100 to train the ML model unsupervised. In other variations, the ML module 124 instructions that function to control the processor 100 to train the ML model supervised using a training dataset with one or more materials tagged with one or more EFOMs. Stated differently, in some variations the input dataset can include one or material representations tagged with an EFOM (e.g., a training dataset) and the ML module 124 trains the ML model to predict the tagged EFOM for the one or more material representations to within a desired value (i.e., less than or equal to a desired value) of a cost function (also known as a “loss function”). And after training of the ML model, the ML module 124 includes instructions that function to control the processor 100 to predict the EFOM for material representations that are not tagged with the EFOM (i.e., not in the training dataset).

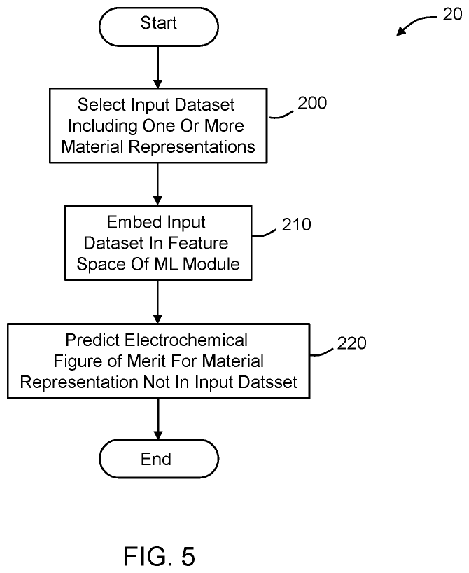

Referring now to FIG. 5, a flow chart for a ML method 20 is shown. The ML method 20 includes selecting an input dataset including one or more material representations comprising electrochemical parameters and one or more stable and/or metastable chemical species from a Pourbaix diagram at 200, embedding the input dataset in a feature space of a ML module at 210, and predicting, based at least in part on the embedded input dataset, an EFOM for a material representation not in the input dataset at 220. In some variations, the ML method 20 includes unsupervised training a ML model with the input dataset before predicting the EFOM.

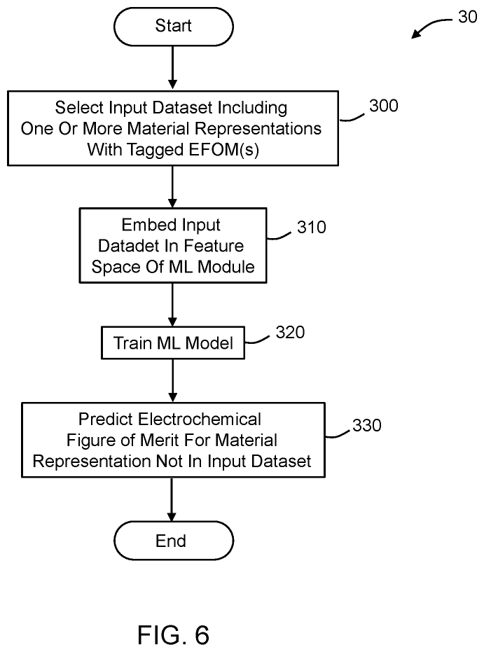

Referring to FIG. 6, a flow chart for another ML method 30 is shown. The ML method 30 includes selecting an input dataset including material representations comprising electrochemical parameters and one or more stable and/or metastable chemical species from a Pourbaix diagram, and one or more known or trusted EFOMs tagged to one or more of the material representations at 300, embedding the input dataset in a feature space of a ML module at 310, and supervised training a ML model at 320. Non-limiting examples of the ML model include a recurrent neural network (RNN), a convolutional neural network (CNN), a Random Forest model, a linear regression model, and combinations thereof. And after the ML model is trained at 320, the method 30 proceeds to predicting, based at least in part on the embedded input dataset, an EFOM for a material representation not in the input dataset at 330.

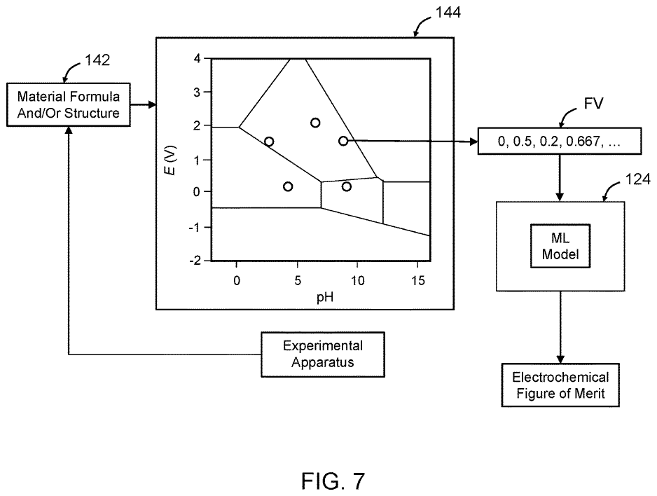

Referring now to FIG. 7, a diagram illustrating the ML method 20 and/or 30 is shown. For example, the acquisition module 122 (FIG. 4) selects a material, e.g., a material formula and/or a material structure, from the candidate dataset 142, and in combination with a Pourbaix diagram selected from the Pourbaix diagram dataset 144, selects electrochemical parameters (i.e., potential and pH) and possible stable and/or metastable chemical species from the Pourbaix diagram to provide and embed a feature vector ‘FV’ (i.e., an input dataset) in a feature space of the ML module 124. In variations where a trained ML model is present in the ML module 124, the ML model predicts, based at least in part on the embedded feature vector FV, one or more EFOMs for the material. In some variations, the ML predicts the one or more electrochemical figures of merit for the material as a function of the Pourbaix diagram parameters. That is, the ML predicts the one or more electrochemical figures of merit for the material as a function of a potential, or a range of potentials, applied to the material and a pH, or a range of pH, for the liquid in which the material is in contact with.

In variations where a trained ML model is not present in the ML module 124, i.e., the ML model is not trained, the ML module 124 trains the ML module with one or more experimentally determined or calculated EFOMS. For example, and as shown in FIG. 7, in some variations an experimental apparatus is used to measure one or more EFOMs for a material and as a function of one or more Pourbaix diagram parameters and such experimentally determined EFOMs are used in combination with the Pourbaix diagram parameters as training data for the ML model. And after the ML model is desirably trained, the ML model predicts, based at least in part on the embedded feature vector, one or more EFOMs for the material as a function of a potential, or a range of potentials, applied to the material and a pH, or a range of pH, for the liquid in which the material is in contact with. Non-limiting examples of an experimental apparatus and/or experimental techniques used to measure one or more electrochemical figures of merit include a scanning droplet cell, differential electrochemical mass spectroscopy (DEMS), inductively coupled plasma mass spectroscopy (ICP-MS), and a potentiotstat, among others.

The preceding description is merely illustrative in nature and is in no way intended to limit the disclosure, its application, or uses. Work of the presently named inventors, to the extent it may be described in the background section, as well as aspects of the description that may not otherwise qualify as prior art at the time of filing, are neither expressly nor impliedly admitted as prior art against the present technology.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple variations or forms having stated features is not intended to exclude other variations or forms having additional features, or other variations or forms incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/−10% of the measured value, while in other variations such known commercial and/or experimental measurement tolerances are +/−5% of the measured value, while in still other variations such known commercial and/or experimental measurement tolerances are +/−2.5% of the measured value. And in at least one variation, such known commercial and/or experimental measurement tolerances are +/−1% of the measured value.

The flowcharts and block diagrams in the figures illustrate the architecture, functionality, and operation of possible implementations of systems, methods, and computer program products according to various embodiments. In this regard, a block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a ROM, an EPROM or flash memory, a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an ASIC, a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, radio frequency (RF), etc., or any suitable combination of the foregoing. Computer program code for carrying out operations for aspects of the present arrangements may be written in any combination of one or more programming languages, including an object-oriented programming language such as Java™, Smalltalk, C++, Python or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one variation, or various variations means that a particular feature, structure, or characteristic described in connection with a form or variation, or particular system is included in at least one variation or form. The appearances of the phrase “in one variation” (or variations thereof) are not necessarily referring to the same variation or form. It should be also understood that the various method steps discussed herein do not have to be carried out in the same order as depicted, and not each method step is required in each variation or form.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. A system comprising:
a processor and a memory communicably coupled to the processor and storing machine-readable instructions that, when executed by the processor, cause the processor to:
embed an input dataset in a feature space of a machine learning module, the input dataset including a material representation comprising a material composition, electrochemical parameters from a Pourbaix diagram, and chemical species of the material composition in contact with a liquid; and
predict, based at least in part on the input dataset embedded in the feature space, an electrochemical figure of merit for the material representation exposed to the liquid.

2. The system according to claim 1 further comprising an acquisition module stored in the memory and including machine-readable instructions that, when executed by the processor, cause the processor to select the input dataset from a candidate dataset and a Pourbaix diagram dataset.

3. The system according to claim 1, wherein the chemical species include at least one of stable chemical species predicted from the Pourbaix diagram.

4. The system according to claim 3, wherein the chemical species further include at least one metastable chemical species derived from the Pourbaix diagram.

5. The system according to claim 4, wherein the electrochemical parameters include electric potential applied to the material composition and pH of the liquid.

6. The system according to claim 1, wherein the electrochemical figure of merit is selected from the group consisting of corrosion rate, catalytic activity, and discharge rate, and combinations thereof.

7. The system according to claim 6 further comprising a machine learning module with machine-readable instructions that, when executed by the processor, cause the processor to train a machine learning model to predict, based at least in part on the input dataset embedded in the feature space, the electrochemical figure of merit for the material composition exposed to the liquid.

8. The system according to claim 7, wherein the machine learning model is selected from the group consisting of a recurrent neural network (RNN), a convolutional neural network (CNN), a Random Forest model, a linear regression model, and combinations thereof.

9. The system according to claim 8, wherein the machine-readable instructions of the machine learning module when executed by the processor, cause the processor to train the machine learning model unsupervised.

10. The system according to claim 8, wherein the machine-readable instructions of the machine learning module when executed by the processor, cause the processor to train the machine learning model supervised.

11. The system according to claim 10, wherein the input dataset further includes an electrochemical figure of merit tagged to the material representation.

12. A system comprising:
a processor; and
a memory communicably coupled to the processor, the memory storing:
an acquisition module and a machine learning module including instructions that when executed by the processor cause the processor to:
select an input dataset from a candidate dataset and a Pourbaix diagram dataset, the input dataset including material representations comprising a material composition, electrochemical parameters from a Pourbaix diagram, and chemical species of the material composition in contact with an aqueous solution;
embed the input dataset in a feature space of the machine learning module;
train a machine learning model during one or more iterations to predict, based at least in part on the input dataset embedded in the feature space, an electrochemical figure of merit for the material representations in the input dataset; and
predict, based at least in part on the training of the machine learning model, the electrochemical figure of merit for a material representation not in the input dataset.

13. The system according to claim 12, wherein the chemical species include at least one of stable chemical species of the material composition in contact with the aqueous solution and predicted from the Pourbaix diagram.

14. The system according to claim 13, wherein the chemical species further include at least one of metastable chemical species of the material composition in contact with the aqueous solution and derived from the Pourbaix diagram.

15. The system according to claim 14, wherein the electrochemical parameters include electric potential applied to the material composition and pH of the aqueous solution.

16. The system according to claim 12, wherein the electrochemical figure of merit is selected from the group consisting of corrosion rate, catalytic activity, and discharge rate, and combinations thereof.

17. The system according to claim 12, wherein at least a portion of the material representations further comprise an electrochemical figure of merit for the material composition exposed to the aqueous solution, and the instructions of the machine learning module, when executed by the processor, cause the processor to supervise train the machine learning model based at least in part on the tagged electrochemical figure of merit.

18. A method comprising:
embedding an input dataset in a feature space of a machine learning module, the input dataset including a material representation comprising a material composition, electrochemical parameters from a Pourbaix diagram, and chemical species of the material composition in contact with a liquid; and
predicting, based at least in part on the input dataset embedded in the feature space, an electrochemical figure of merit for another material representation not in the input dataset.

19. The method according to claim 18, wherein the chemical species includes at least one of stable chemical species predicted from the Pourbaix diagram and metastable chemical species derived from the Pourbaix diagram.

20. The method according to claim 18 further comprising training a machine learning model to predict, based at least in part on the input dataset embedded in the feature space, the electrochemical figure of merit for the material composition in the input dataset.
